(19) United States
(12) Patent Application Publication
(10) Pub. No.: US 2005/0031670 A1
(43) Pub. Date: Feb. 10, 2005

(54) Weight reduction or weight controlling composition

(76) Inventors: Brenda Diane Jamerson, Research Triangle Park, NC (US); Alan Metz, Durham, NC (US); Mickey Lee Wells, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/478,227

(22) PCT Filed: May 20, 2002
(86) PCT No.: PCT/US02/15871

Related U.S. Application Data
(60) Provisional application No. 60/292,634, filed on May 22, 2001.

(51) Int. Cl. A61K 31/537; A61K 47/00

(52) U.S. Cl. 424/439; 514/237.5

(57) ABSTRACT

A composition having weight reduction capability, weight controlling activity, or both comprising (a) a bupropion compound and (b) a food component and a method of treatment associated with it.

### FIELD OF THE INVENTION

The present invention relates to a pharmaceutical composition having weight reduction and/or weight controlling activity comprising (a) a bupropion compound and (b) a nutritional, calorie-limited food or beverage component.

### BACKGROUND OF THE INVENTION

Overweight and obesity are an increasing health problem. Both conditions have been associated with them increased mortality from a number of causes, especially cardiovascular disease.

A large percentage of obese and overweight people fail to benefit from available dietary, behavioral and pharmacological treatments used for weight reduction. When used alone, these treatments typically result in a modest weight loss.

Weight loss is generally achieved by restricting the calories needed to maintain a certain body weight. Therefore, when required to limit food intake to a predetermined caloric amount, such as 800 to 2300 calories per day, many people are unable to comply consistently on a daily basis. A major problem with such diets is that they involve the necessity for some arithmetic in calculating how many calories and how many grams of each type of food may be eaten in a particular day. This calculation can be irritating for the dieter. It is also difficult to monitor diet compliance from the perspective of the physician or monitoring dietician. Such diets are time consuming, tedious, and oftentimes boring. Many low calorie diets, including diet beverages and snack bars, are not hunger-abating or give the dieter a feeling of satiety. Accordingly, those attempting to lose or control weight in this manner often gain the weight back over time.

In general, weight loss and weight control agents may be classified as satiety agents, lipase inhibitors, neurotransmitter re-uptake inhibitors, adrenergics, cannabinoid antagonists, or a ciliary neurotrophic factor. There are no combinations of diet and centrally acting agent(s) that have been proven effective for weight loss. Centrally acting agent(s) are ones that act on the central nervous system, especially on the brain and/or spinal cord.

### SUMMARY OF THE INVENTION

In accordance with the present invention, it is believed that centrally acting agents aid the dieter in achieving weight loss by (1) aiding compliance to a calorie restricted diet and (2) by allowing the dieter to feel more “satisfied” with fewer calories, presumably by action at norepinephrine and dopaminergic sites in the brain. It is therefore an object of the invention to provide a complete, hunger-abating, low-calorie, and/or high energy edible composition. The present invention meets this on-going need by providing a composition having weight reduction, weight controlling activity or both comprising (a) a bupropion compound and (b) a food component. Preferably the food component is nutritional and contains a limited or restricted number of calories. There is also provided a method for weight reduction, weight control, and/or appetite suppression comprising the the administration of the above-identified composition to a mammal, preferably a human.

### DETAILED DESCRIPTION OF THE INVENTION

As used herein, “bupropion compound(s)” is bupropion, a salt of bupropion, a solvate of bupropion or of its salt, a metabolite of bupropion, a prodrug of any of these, and enantioisomers and/or diesterioisomers of any of these compounds. Bupropion (especially its hydrochloric salt, bupropion HCl) is a well-known antidepressant and smoking cessation drug that is commercially available as Wellbutrin™ or Zyban™. This active drug substance is a racemate, chemically known as (±)-1-(3-chlorophenyl)-2-[(1,1-dimethyl)amino]-1-propanone hydrochloride. In the invention herein, it is preferably employed in a controlled release form (e.g., sustained release (SR), extended release (ER), delayed release, modified release, long-lasting release, continuous release, and/or a controlled release formulation). Such formulations are known and can include, for example, osmotic dosage forms, multiple particulate dosage forms (including prills), use of swellable and non-swellable polymers, effervescent agents, use of pore-forming and non-pore forming agents, use of gelatinous dispersions, use of pulsatile drug delivery systems, use of multi-coated tablets, use of semi-permeable membranes and permeable membranes, use of channeling-leaching agents, use of coating agents, and the like). Instant release formulations of a bupropion compound can be used in the invention but may be generally less desirable because of a possible an increased risk of seizures. Bupropion is well known and disclosed, for example, in U.S. Pat. Nos. 3,885,046; 3,819,706; 5,358,970; 5,541,231; 5,731,000; 5,763,493; 5,969,553; 5,427,798; RE 33,994 (reissue of 4,687,660); and WO 94/04138; WO 99/33457; WO 00/30685. Bupropion HCl is disclosed in the Merck Index and the Physician's Desk Reference and the chemical name for bupropion HCl is (+)-2-(tert-butylamino)-3′-chloropropiophenome hydrochloride.

Other active compounds suitable for use as the bupropion compound are disclosed in U.S. Pat. No. 6,274,579 to Morgan et al. (especially (+)-(2S,3S)-2-(3-chlorophenyl)-3,5,5-trimethyl-2-morpholinol and pharmaceutically salts and solvates thereof), CA 2,259,730; WO 94/04138; WO 99/25355; and WO 01/62257 (especially (R,R)-2(3-chlorophenyl-2-hydroxy-3,5,5,-trimethyl-morphinol). The morpholinol metabolite (+/−)-(2R*,3R*)-2-(3-chlorophenyl)-3,5,5-trimethyl-2-morpholinol hydrochloride can also be employed as the bupropion compound herein. Particularly preferred compounds are bupropion hydrochloride and (+)-(2S,3S)-2-(3-chlorophenyl)-3,5,5-trimethyl-2-morpholinol and pharmaceutically acceptable salts and solvates thereof.

In the pharmaceutical composition of the invention comprising a bupropion compound and a nutritional, calorie-limited food component (beverage or solid such as a snack bar), the bupropion compound can be free of stabilization or can be stabilized using any number of known methods of stabilization. Such methods are taught in some of the above-described references or are disclosed in U.S. Pat. Nos. 6,096,341; 6,143,327; 6,033686; 5,558,879; 5,968,553; 5,472,708; 5,508,040; 6,210,716; and WO 00/50010 and WO 00/30685. Preferably, the bupropion compound, e.g., bupropion HCl, is stabilized. As used herein, the term stabilizer means a composition which inhibits or prevents the decomposition of the bupropion compound (e.g., bupropion HCl). In general stabilization is accomplished in the art using one or more inorganic acids, organic acids, and/or using core/shell, coating, or encapsulating techniques. Such techniques and formulations are disclosed, for example, in U.S. Pat. Nos. 5,427,798 and 5,358,970. Preferably, stabilizers suitable for use are those which have an aqueous solution pH of about 0.9 to about 4 at an aqueous solution concentration of about 6% W/w and are a solid or liquid at 30 degrees Centigrade as determined by the procedure described in U.S. Pat. No. 5,358,970. Stabilizers which are useful in this invention include: L-cysteine hydrochloride, glycine hydrochloride, ascorbic acid, malic acid, sodium metabisulfite, isoascorbic acid, citric acid, alginic acid, tartaric acid, L-cystine dihydrochloride, and mixtures thereof. L-cysteine hydrochloride, glycine hydrochloride, and alginic acid are most preferred.

In the composition of the invention the bupropion compound is present in an amount ranging from about 10 mg to 600 mg, preferably 50 to 500 mg, most preferably 100 to 350 mg.

The food component can be a beverage, a snack bar, or one or more powders. The food component of the invention, in general, contains protein, carbohydrate, fat, vitamins and minerals. Preferably the amount of vitamins and minerals is the recommended allowance of minerals and vitamins. Typically, the food component can contain from 0 to 600 calories, preferably 0 to 450 calories, most preferably from about 0 to 300 calories.

Generally, this component comprises about 5 to 75 grams, preferably 5 to 45 grams, and most preferably 5 to 30 grams of protein. The protein can be provided by milk or a milk product (e.g., whey protein, dry milk). It is further preferred, however, that all or a portion of the protein be comprised of vegetable protein, for example, soy protein, for it is believed that vegetable protein reduces levels of cholesterol in the blood, thereby lowering the risk of heart disease in the consumer.

The carbohydrate is present in this component in an amount ranging from about 20 to 180 grams, preferably about 20 to about 60 grams, and most preferably about 25 to 50 grams. The carbohydrate is preferably provided from milk, powdered milk or milk product or fruit juice of fruit extract (dry or liquid), sugar or other natural or artificial sweetner.

The food component has a fat content ranging from about 1 to 30 grams, preferably from about 3 to 15 grams, most preferably from about 3 to about 10 grams. Further the fat in the component may contain at least 10% medium-chain triglycerides (MCTs), at level at least 10% of the total fat in the diet, as this may be effective in reducing body adipose tissue.

In general, each serving of the composition of the invention may contain vitamins and minerals. Such vitamins and minerals can include, for example, vitamins A, C, D, E, B6, B12, K, riboflavin, niacin, thiamin, pantothenic acid, biotin, folate, calcium, iron, iodine, zinc, manganese, molybdenum, chromium, selenium, magnesium, phosphorus. Typically, the amounts of each vitamin and/or mineral may be as follows: about 25-50% Vitamin A; 50-150% Vitamin C; 25-50% Vitamin D; 50-150% Vitamin E; 10-50% Vitamin K; 25-50% Vitamin B6; 20-60% Vitamin B12; 25-60% calcium; 5-45% iron; 20-50% riboflavin; 20-50% thiamin; 20-50% niacin; 20-45% molybdenum; 20-55% manganese; 5-50% zinc; 10-45% iodine; 10-45% chromium; 5-40% selenium; 10 65% magnesium; 20-60% phosphorus; 10-45% folate; 10-55% panothenic acid; and 10-50% biotin. A most preferred vitamin and mineral regimen contains: 35% Vitamin A, 100% Vitamin C, 35% Vitamin D, 100% Vitamin E, 40% calcium, 15% iron, 25% Vitamin K, 35% thiamin, 35% riboflavin, 35% niacin, 35% Vitamin B6, 35% Vitamin B12, 35% pantothenic acid, 35% iodine, 15% zinc, 35% chromium, 35% manganese, 30% folate, 35% biotin, 35% molybdenum, 35% magnesium, 25% selenium, and 40% phosphorus. (The above are percent daily values based upon a 2,000 diet, and may be higher or lower depending on individual calorie needs.) Further, the composition of the invention can contain 500 to 650 mg potassium.

It is desirable that the composition of the invention be low in cholesterol (less than 30 mg) and sodium (less than 700 mg), preferably containing 0 to 10 mg cholesterol and less than 350 mg sodium.

Additionally, the food component can optionally, and preferably does, contain fiber, typically 1 to 10 grams of fiber per 200 to 1200 calories (Kcal.) size serving, preferably 3 to 6 grams of fiber for every 200 to 300 calories. Preferably, the component contains fiber known as “slow release” fiber, that is fiber coated with any substance that resists saliva but breaks down when contacted with gastric juices, such that the fiber is release slowly in the stomach. Generally this takes the form of guar gum coated with a protein that can be broken down by stomach/gastric juices. This type of fiber provides palatable fiber and may aid or prevent the phenomenon of “insulin rebound.”

The amino acid tryptophan may also be included to provide a sense of satiety. This amino acid may be converted by the body to the neurotransmitter serotonin which may play a role in controlling appetite. When employed, it is present in an amount ranging from about 1 to 5 grams. Other satiety agents may also be advantageous employed in the composition of the invention.

It is usually desirable to include at least one flavoring agent as well as a sugar or non-nutritive sweetener. Non-limiting examples of such sugars or sweeteners include sugar, fructose, lactose, corn syrup, honey, aspartame, saccharin, licorice root extracts, other food-grade sweeteners, and mixtures thereof. Of these, non-nutritive sweeteners are preferred. Non-limiting examples of flavoring agents (natural and/or artificial) that may be used include any of the food grade flavoring agents suitable for use in beverages and/or snack bars. These can include, for example, fruit flavors (e.g., strawberry, banana, orange, raspberry, grape, lemon-lime, etc.), chocolate, vanilla, mint, root beer, cola, hazelnut, ginger, and mixtures of them. Generally, the flavoring is used in an effective amount, one that will flavor the snack bar or beverage, but not so much as to overwhelm the composition. This amount will generally not exceed about 0.2 wt %, based on the total weight of the beverage or snack bar. A sweetener or sugar will generally be used in an amount of about 0.025 wt. % to about 30 wt. % but, of course, it can be adjusted to any level of sweetness desired while keeping within the calorie requirement of the entire beverage or snack bar.

If desired, a preservative may also be used. Food-grade preservatives that can be employed in the invention can include, for example, antimicrobial preservatives such as bezoic acid, sodium benzoate, EDTA, sorbic acid, potassium sorbate, methylparaben, propylparaben, butylparatben; antioxidant preservatives such as ascorbic acid, fumaric acid, malic acid, alpha tocopherol, butylated hyrodroxyanisole (BHA), and butylated hydroxytoluene (BHT).

The composition of the invention can take the form of a dietary beverage, snack bar, or powdered formulation. Alternatively, a tablet of a bupropion compound can be administered along with a dietary beverage or snack bar as described herein. Such dietary beverages or snack bars are readily commercially available. Preferably, the present invention is a formulation in the form of one or more powders, preferably two powders that can be added to a liquid. Most preferably, the bupropion compound and the nutritional food component may be combined in one powder. When two powders are employed one powder can comprise the bupropion compound and the other can contain the food component. The two powders are combined in water, milk, a milk substitute (e.g., one based on soy or other cereal legumes, rice, whey, etc.), juice, soda, or a like liquid. Of course, mixtures of these liquids can be employed. The powders and liquid can be combined in any order. However, it is preferable to add the powder containing the bupropion compound last. The powders can be placed in an appropriate container for the liquid and mixed by stirring, shaking, and/or blending. Alternatively, the bupropion compound can be mixed or combined in a snack bar or served as a tablet/capsule along with a snack bar.

As a commercial preparation, the beverage product of the present invention can be a dry blend, a concentrate, or a liquid beverage. The dry blend can be prepared by any suitable method. One suitable method is to merely blend the ingredients and drying them to the appropriate or desired moisture level by air, vacuum, or spray drying. Prior to drying, the ingredients, preferably blended, can be heated (at temperatures between about 170 degrees F. to 300 degrees F.) and/or pasteurized. The dry blend is then sealed in an appropriate packet or container. These blended components can be added to any suitable/desired liquid such as water, milk (e.g., non-fat skim milk), soda, and/or fruit juice. It will be noted that milk based beverages are typically homogenized first and then pasteurized using conventional techniques. Pasterization/homogenation is conducted at pressures from about 2,000 to 14,000 psig, preferably 2,000 to 14,000 psig, most preferably 2,000 to 8,000 psig. The homogenation will be conducted for a period of time to effect homogenization of the components of the mixture. While ambient temperatures are preferred during the homogenization, it is understood that the temperature can range from about 120 degrees F. to about 300 degrees F., preferably from about 140 to about 200 degrees F.

The beverage, powdered formulation, or snack bar containing the bupropion compound and food component are administered or ingested preferably within one hour of container opening or within one hour of blending the bupropion into a liquid in the case of a beverage. Preferably the composition of the invention is substituted for two meals per day and the dieter ingests a regular meal averaging 200-1500 calories, preferably 250 to 600 calories, most preferably 250 to 400 calories, in the evening.

All references mentioned in this document are hereby incorporated by reference.

### EXPERIMENTAL

Example 1 (Comparative): Obese patients (BMI≧30) were given a food component alone (i.e., a low-calorie, nutritional beverage (Slim Fast™, 325 mL) for two meals per day for 6 months plus one normal evening meal to achieve a total 600 Kcal deficit diet for the day. After 6 months, the average weight loss per patient was 5 kg for those completing the program.

Example 2 (Comparative): An analysis was conducted of obese patients (BMI≧30) who were given 300 mg of bupropion (150 mg twice a day) in the absence of the food component of the invention nor any other dietary counseling or intervention. Weight loss was negligible, the average weight loss per patient in the 300 mg study was 2.4 Kg after 12 months.

Example 3 (Invention): Obese patients (BMI≧30) were given 300 mg (N=110) or 400 mg (N=105) of bupropion and a low calorie, nutritional beverage (Slim Fast™, 11.5 oz.) for two meals per day for 6 months to achieve a 600 Kcal deficit diet. That is, each patient was given 150 or 200 mg of bupropion HCL sustained release, respectively, in a nutritional beverage (a food component of the invention) twice a day. At the end of the end of 6 months, the average weight loss was 7.2 kg and 10.1 kg for the 300 mg and 400 mg groups, respectively.

1. A composition having weight reduction capability, weight controlling activity, or both comprising (a) a bupropion compound and (b) a food component.

2. The composition of claim 1 wherein the food component comprises at least one ingredient selected from the group consisting of protein, carbohydrate, fat, minerals, and vitamins.

3. The composition of claim 2 wherein the bupropion compound is present in an amount ranging from about 10 mg to about 600 mg; the protein is present in an amount ranging from about 5 grams to 75 grams; the carbohydrate is present in an amount ranging from about 20 grams to 180 grams; and the fat is present in an amount ranging from about 1 gram to about 30 grams.

4. The composition of claim 1 wherein the composition contains at least one ingredient selected from the group consisting of (i) fiber, (ii) flavoring agent, (iii) preservative, (iv) a sugar, and (v) a non-nutritive sweetener.

5. The composition of claim 1 wherein the bupropion compound is selected from the group consisting of an instant release formulation, a controlled release formulation, and a mixture thereof.

6. The composition of claim 1 wherein the bupropion compound is selected from the group consisting of (i) a bupropion HCl instant release form, (ii) a bupropion HCl sustained release form, (iii) (+)-(2S,3S)-2-(3-chlorophenyl)-3,5,5-trimethyl-2-morpholinol and pharmaceutically salts and solvates thereof, and (iv) a mixture thereof.

7. The composition of claim 1 in the form of a beverage, snack bar, or one or more powders.

8. The composition of claim 1 wherein the bupropion compound is bupropion HCl sustained release and the food component contains 10 grams of protein, 40 grams of carbohydrate, 3 grams of fat, 5 grams of fiber, vitamins and minerals, a flavoring, and optionally a preservative.

9. The composition of claim 1 wherein the bupropion compound is stabilized by a compound selected from the group consisting of L-cysteine hydrochloride, glycine hydrochloride, ascorbic acid, malic acid, sodium metabisulfite, isoascorbic acid, citric acid, alginic acid, tartaric acid, L-cystine dihydrochloride, and mixtures thereof.

10. A method for the treatment of at least one selected from the group consisting of weight reduction, weight control, and appetite suppression comprising the administration of the composition of claim 1 to a mammal.

11. The method of claim 10 wherein the food component comprises at least one ingredient selected from the group consisting of protein, carbohydrate, fat, minerals, and vitamins.

12. The method of claim 10 wherein the bupropion compound is present in an amount ranging from about 10 mg to about 500 mg; the protein is present in an amount ranging from about 5 grams to 75 grams; the carbohydrate is present in an amount ranging from about 20 grams to 180 grams; and the fat is present in an amount ranging from about 1 gram to about 30 grams.

13. The method of claim 10 wherein the composition contains at least one ingredient selected from the group consisting of (i) fiber, (ii) flavoring agent, (iii) preservative, (iv) a sugar, and (v) a non-nutritive sweetener.

14. The method of claim 10 wherein the bupropion compound is selected from the group consisting of an instant release formulation, a controlled release formulation, and a mixture thereof.

15. The method of claim 10 wherein the bupropion compound is selected from the group consisting of (i) a bupropion HCl instant release form, (ii) a bupropion HCl sustained release form, (iii) (+)-(2S,3S)-2-(3-chlorophenyl)-3,5,5-trimethyl-2-morpholinol and pharmaceutically salts and solvates thereof, and (iv) a mixture thereof; and the food component contains 10 grams of protein, 40 grams of carbohydrate, 3 grams of fat, 5 grams of fiber, vitamins and minerals, a flavoring, and optionally a preservative.

16. The method of claim 10 wherein the mammal is a human.

17. The method of claim 10 wherein the bupropion compound is stabilized by a compound selected from the group consisting of L-cysteine hydrochloride, glycine hydrochloride, ascorbic acid, malic acid, sodium metabisulfite, isoascorbic acid, citric acid, alginic acid, tartaric acid, L-cystine dihydrochloride, and mixtures thereof.
